(19) United States
(12) Patent Application Publication
(10) Pub. No.: US 2026/0315796 A1
(43) Pub. Date: Oct. 15, 2026

(54) SYSTEMS AND METHODS FOR IDENTIFYING AN EVOKED RESPONSE MEASURED THROUGH A COCHLEAR IMPLANT

(71) Applicant: Advanced Bionics LLC, Valencia, CA (US)

(72) Inventors: Chen Chen, Valencia, CA (US); Anthony J. Spahr, Newhall, CA (US); Sridhar Kalluri, Pasadena, CA (US); Phillipp Hehrmann, Hannover (DE); Raphael S. Koning, Wedemark (DE); Mehrangiz Ashiri, Winnipeg (CA)

(21) Appl. No.: 19/485,552

(22) PCT Filed: Sep. 8, 2023
(86) PCT No.: PCT/US2023/032286

§ 371 Date: Nov. 18, 2025

Related U.S. Application Data
(60) Provisional application No. 63/525,281, filed on Jul. 6, 2023.

(51) Int. Cl. A61N 1/36; A61N 1/05

(52) U.S. Cl. CPC A61N 1/36038; A61N 1/0541

(57) ABSTRACT

An exemplary system comprises a memory storing instructions and a processor configured to execute the instructions to perform a process. The process may comprise directing stimulation to be applied to a recipient of a hearing system, the stimulation configured to elicit an evoked response within the recipient, directing a cochlear implant included in the hearing system to continuously record, during an acquisition time window and using at least one electrode electrically coupled to the cochlear implant, data representative of the evoked response, and directing the cochlear implant to stream, by way of a back telemetry channel, the data to a computing device external to the recipient during the acquisition time window while the cochlear implant is continuously recording the data.

### RELATED APPLICATIONS

The present application claims priority to U.S. Provisional Patent Application No. 63/525,281, filed Jul. 6, 2023, the contents of which is hereby incorporated by reference in its entirety.

### BACKGROUND INFORMATION

Cochlear implant systems are used to provide, restore, and/or improve the sense of hearing to recipients with severe or profound hearing loss. Conventional cochlear implant systems include various components configured to be implanted within a recipient (e.g., an electronics package, an antenna, and an electrode lead) and various components configured to be located external to the recipient (e.g., a sound processor, a battery, and a microphone).

An electrode lead of a cochlear implant system may include an electrode array comprised of metal electrode contacts (e.g., platinum, titanium, etc.) insulated by the medical grade silicone. The metal electrode contacts are configured to be inserted within a cochlea of a recipient and are typically used to apply electrical stimulation to one or more intracochlear locations. When stimulation is applied to the recipient of a cochlear implant system, the metal electrode contacts on the electrode array may also be used to measure an evoked response that occurs within the recipient based on the stimulation. However, measuring an evoked response through a cochlear implant is difficult due to challenging morphology and because the measured signals have a long acquisition window and a relatively small amplitude with competing artifacts and/or background noise. In addition, the implanted components of a cochlear implant system typically have limited buffer storage space for data associated with such measurements, which makes it difficult to adequately collect information indicative of evoked responses in a clinically viable manner.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary cochlear implant system.

FIG. 2 shows an exemplary configuration of the cochlear implant system of FIG. 1.

FIG. 3 shows another exemplary configuration of the cochlear implant system of FIG. 1.

FIG. 4 shows an exemplary evoked response detection system according to principles described herein.

FIG. 5 shows an exemplary configuration of the evoked response detection system of FIG. 4 according to principles described herein.

FIG. 6 is an exemplary flow diagram that depicts exemplary operations that may be performed according to principles described herein.

FIGS. 7-10 depict additional exemplary configurations of the evoked response detection system of FIG. 4 according to principles described herein.

FIG. 11 shows an exemplary method for identifying an evoked response measured through a cochlear implant according to principles described herein.

FIG. 12 shows an exemplary computing device according to principles described herein.

### DETAILED DESCRIPTION

Systems and methods for identifying an evoked response measured through a cochlear implant are described herein. An exemplary system comprises a memory storing instructions and a processor configured to execute the instructions to perform a process. The process may comprise directing stimulation to be applied to a recipient of a hearing system, the stimulation configured to elicit an evoked response within the recipient, directing a cochlear implant included in the hearing system to continuously record, during an acquisition time window and using at least one electrode electrically coupled to the cochlear implant, data representative of the evoked response, and directing the cochlear implant to stream, by way of a back telemetry channel, the data to a computing device external to the recipient during the acquisition time window while the cochlear implant is continuously recording the data.

An additional exemplary system comprises a cochlear implant and at least one electrode electrically coupled to the cochlear implant. In such a system, the cochlear implant may be configured to continuously record, during an acquisition time window and using the at least one electrode electrically coupled to the cochlear implant, data representative of an evoked response that occurs within a recipient of the cochlear implant in response to stimulation applied to the recipient, and stream, by way of a back telemetry channel, the data to a computing device external to the recipient during the acquisition time window while the cochlear implant is continuously recording the data.

The systems and methods described herein may provide various benefits to cochlear implant recipients, as well as others involved with managing cochlear implant systems. For example, systems and methods such as those described herein may facilitate efficiently transmitting data indicative of evoked responses and automatically identifying key features (e.g., peaks, amplitudes, etc.) of the evoked responses in a clinically effective manner that minimizes the analytical burden of a clinician. Systems and methods such as those described herein may also assist a clinician in evaluating one or more conditions (e.g., a residual hearing status) of the recipient, and/or otherwise provide benefit to the recipient. For example, systems and methods such as those described herein are especially beneficial for infant cochlear implant recipients that are unable to give feedback about their hearing.

Various embodiments will now be described in more detail with reference to the figures. The disclosed systems and methods may provide one or more of the benefits mentioned above and/or various additional and/or alternative benefits that will be made apparent herein.

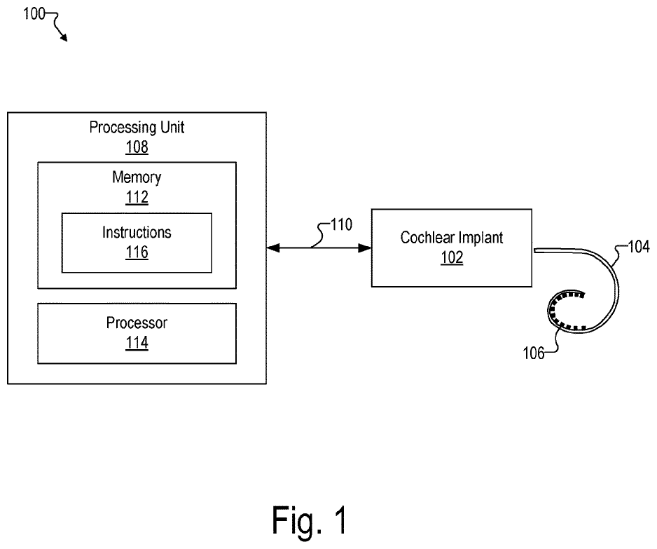

FIG. 1 illustrates an exemplary cochlear implant system 100 configured to be used by a recipient. As shown, cochlear implant system 100 includes a cochlear implant 102, an electrode lead 104 physically coupled to cochlear implant 102 and having an array of electrodes 106, and a processing unit 108 configured to be communicatively coupled to cochlear implant 102 by way of a communication link 110.

The cochlear implant system 100 shown in FIG. 1 is unilateral (i.e., associated with only one ear of the recipient). Alternatively, a bilateral configuration of cochlear implant system 100 may include separate cochlear implants and electrode leads for each ear of the recipient. In the bilateral configuration, processing unit 108 may be implemented by a single processing unit configured to interface with both cochlear implants or by two separate processing units each configured to interface with a different one of the cochlear implants.

Cochlear implant 102 may be implemented by any suitable type of implantable stimulator. For example, cochlear implant 102 may be implemented by an implantable cochlear stimulator. Additionally or alternatively, cochlear implant 102 may be implemented by a brainstem implant and/or any other type of device that may be implanted within the recipient and configured to apply electrical stimulation to one or more stimulation sites located along an auditory pathway of the recipient.

In some examples, cochlear implant 102 may be configured to generate electrical stimulation representative of an audio signal processed by processing unit 108 in accordance with one or more stimulation parameters transmitted to cochlear implant 102 by processing unit 108. Cochlear implant 102 may be further configured to apply the electrical stimulation to one or more stimulation sites (e.g., one or more intracochlear locations) within the recipient by way of one or more electrodes 106 on electrode lead 104. In some examples, cochlear implant 102 may include a plurality of independent current sources each associated with a channel defined by one or more of electrodes 106. In this manner, different stimulation current levels may be applied to multiple stimulation sites simultaneously by way of multiple electrodes 106.

Cochlear implant 102 may additionally or alternatively be configured to generate, store, and/or transmit data. For example, cochlear implant may use one or more electrodes 106 to record one or more signals (e.g., one or more voltages, impedances, evoked responses within the recipient, and/or other measurements) and transmit, by way of communication link 110, data representative of the one or more signals to processing unit 108. In some examples, this data is referred to as back telemetry data.

Electrode lead 104 may be implemented in any suitable manner. For example, a distal portion of electrode lead 104 may be pre-curved such that electrode lead 104 conforms with the helical shape of the cochlea after being implanted. Electrode lead 104 may alternatively be naturally straight or of any other suitable configuration.

In some examples, electrode lead 104 includes a plurality of wires (e.g., within an outer sheath) that conductively couple electrodes 106 to one or more current sources within cochlear implant 102. For example, if there are n electrodes 106 on electrode lead 104 and n current sources within cochlear implant 102, there may be n separate wires within electrode lead 104 that are configured to conductively connect each electrode 106 to a different one of the n current sources. Exemplary values for n are 8, 12, 16, or any other suitable number.

Electrodes 106 are located on at least a distal portion of electrode lead 104. In this configuration, after the distal portion of electrode lead 104 is inserted into the cochlea, electrical stimulation may be applied by way of one or more of electrodes 106 to one or more intracochlear locations. One or more other electrodes (e.g., including a ground electrode, not explicitly shown) may also be disposed on other parts of electrode lead 104 (e.g., on a proximal portion of electrode lead 104) to, for example, provide a current return path for stimulation current applied by electrodes 106 and to remain external to the cochlea after the distal portion of electrode lead 104 is inserted into the cochlea. Additionally or alternatively, a housing of cochlear implant 102 may serve as a ground electrode for stimulation current applied by electrodes 106.

Processing unit 108 may be configured to interface with (e.g., control and/or receive data from) cochlear implant 102. For example, processing unit 108 may transmit commands (e.g., stimulation parameters and/or other types of operating parameters in the form of data words included in a forward telemetry sequence) to cochlear implant 102 by way of communication link 110. Processing unit 108 may additionally or alternatively provide operating power to cochlear implant 102 by transmitting one or more power signals to cochlear implant 102 by way of communication link 110. Processing unit 108 may additionally or alternatively receive data from cochlear implant 102 by way of communication link 110. Communication link 110 may be implemented by any suitable number of wired and/or wireless bidirectional and/or unidirectional links.

As shown, processing unit 108 includes a memory 112 and a processor 114 configured to be selectively and communicatively coupled to one another. In some examples, memory 112 and processor 114 may be distributed between multiple devices and/or multiple locations as may serve a particular implementation.

Memory 112 may be implemented by any suitable non-transitory computer-readable medium and/or non-transitory processor-readable medium, such as any combination of non-volatile storage media and/or volatile storage media. Exemplary non-volatile storage media include, but are not limited to, read-only memory, flash memory, a solid-state drive, a magnetic storage device (e.g., a hard drive), ferroelectric random-access memory (“RAM”), and an optical disc. Exemplary volatile storage media include, but are not limited to, RAM (e.g., dynamic RAM).

Memory 112 may maintain (e.g., store) executable data used by processor 114 to perform one or more of the operations described herein. For example, memory 112 may store instructions 116 that may be executed by processor 114 to perform any of the operations described herein. Instructions 116 may be implemented by any suitable application, program (e.g., sound processing program), software, code, and/or other executable data instance. Memory 112 may also maintain any data received, generated, managed, used, and/or transmitted by processor 114.

Processor 114 may be configured to perform (e.g., execute instructions 116 stored in memory 112 to perform) various operations with respect to cochlear implant 102.

To illustrate, processor 114 may be configured to control an operation of cochlear implant 102. For example, processor 114 may receive an audio signal (e.g., by way of a microphone communicatively coupled to processing unit 108, a wireless interface (e.g., a Bluetooth interface), and/or a wired interface (e.g., an auxiliary input port)). Processor 114 may process the audio signal in accordance with a sound processing program (e.g., a sound processing program stored in memory 112) to generate appropriate stimulation parameters. Processor 114 may then transmit the stimulation parameters to cochlear implant 102 to direct cochlear implant 102 to apply electrical stimulation representative of the audio signal to the recipient.

In some implementations, processor 114 may also be configured to apply acoustic stimulation to the recipient. For example, a receiver (also referred to as a loudspeaker) may be optionally coupled to processing unit 108. In this configuration, processor 114 may deliver acoustic stimulation to the recipient by way of the receiver. The acoustic stimulation may be representative of an audio signal (e.g., an amplified version of the audio signal), configured to elicit an evoked response within the recipient, and/or otherwise configured. In configurations in which processor 114 is configured to both deliver acoustic stimulation to the recipient (e.g., at a left ear) and direct cochlear implant 102 to apply electrical stimulation to the recipient (e.g., at a right ear), cochlear implant system 100 may be referred to as a bimodal hearing system and/or any other suitable term.

Processor 114 may be additionally or alternatively configured to receive and process data generated by cochlear implant 102. For example, processor 114 may receive data representative of a signal recorded by cochlear implant 102 using one or more electrodes 106 and, based on the data, adjust one or more operating parameters of processing unit 108. Additionally or alternatively, processor 114 may use the data to perform one or more diagnostic operations with respect to cochlear implant 102 and/or the recipient.

Other operations may be performed by processor 114 as may serve a particular implementation. In the description provided herein, any references to operations performed by processing unit 108 and/or any implementation thereof may be understood to be performed by processor 114 based on instructions 116 stored in memory 112.

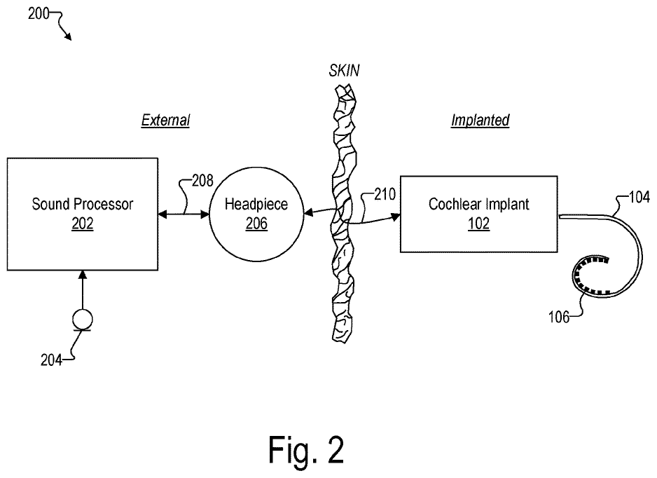

Processing unit 108 may be implemented by one or more devices configured to interface with cochlear implant 102. To illustrate, FIG. 2 shows an exemplary configuration 200 of cochlear implant system 100 in which processing unit 108 is implemented by a sound processor 202 configured to be located external to the recipient. In configuration 200, sound processor 202 is communicatively coupled to a microphone 204 and to a headpiece 206 that are both configured to be located external to the recipient.

Sound processor 202 may be implemented by any suitable device that may be worn or carried by the recipient. For example, sound processor 202 may be implemented by a behind-the-ear (“BTE”) unit configured to be worn behind and/or on top of an ear of the recipient. Additionally or alternatively, sound processor 202 may be implemented by an off-the-ear unit (also referred to as a body worn device) configured to be worn or carried by the recipient away from the ear. Additionally or alternatively, at least a portion of sound processor 202 is implemented by circuitry within headpiece 206.

Microphone 204 is configured to detect one or more audio signals (e.g., that include speech and/or any other type of sound) in an environment of the recipient.

Microphone 204 may be implemented in any suitable manner. For example, microphone 204 may be implemented by a microphone that is configured to be placed within the concha of the ear near the entrance to the ear canal, such as a T-MIC™ microphone from Advanced Bionics. Such a microphone may be held within the concha of the ear near the entrance of the ear canal during normal operation by a boom or stalk that is attached to an ear hook configured to be selectively attached to sound processor 202. Additionally or alternatively, microphone 204 may be implemented by one or more microphones in or on headpiece 206, one or more microphones in or on a housing of sound processor 202, one or more beam-forming microphones, and/or any other suitable microphone as may serve a particular implementation.

Headpiece 206 may be selectively and communicatively coupled to sound processor 202 by way of a communication link 208 (e.g., a cable or any other suitable wired or wireless communication link), which may be implemented in any suitable manner. Headpiece 206 may include an external antenna (e.g., a coil and/or one or more wireless communication components) configured to facilitate selective wireless coupling of sound processor 202 to cochlear implant 102. Headpiece 206 may additionally or alternatively be used to selectively and wirelessly couple any other external device to cochlear implant 102. To this end, headpiece 206 may be configured to be affixed to the recipient's head and positioned such that the external antenna housed within headpiece 206 is communicatively coupled to a corresponding implantable antenna (which may also be implemented by a coil and/or one or more wireless communication components) included within or otherwise connected to cochlear implant 102. In this manner, stimulation parameters and/or power signals may be wirelessly and transcutaneously transmitted between sound processor 202 and cochlear implant 102 by way of a wireless communication link 210.

In configuration 200, sound processor 202 may receive an audio signal detected by microphone 204 by receiving a signal (e.g., an electrical signal) representative of the audio signal from microphone 204. Sound processor 202 may additionally or alternatively receive the audio signal by way of any other suitable interface as described herein. Sound processor 202 may process the audio signal in any of the ways described herein and transmit, by way of headpiece 206, stimulation parameters to cochlear implant 102 to direct cochlear implant 102 to apply electrical stimulation representative of the audio signal to the recipient.

In an alternative configuration, sound processor 202 may be implanted within the recipient instead of being located external to the recipient. In this alternative configuration, which may be referred to as a fully implantable configuration of cochlear implant system 100, sound processor 202 and cochlear implant 102 may be combined into a single device or implemented as separate devices configured to communicate one with another by way of a wired and/or wireless communication link. In a fully implantable implementation of cochlear implant system 100, headpiece 206 may not be included and microphone 204 may be implemented by one or more microphones implanted within the recipient, located within an ear canal of the recipient, and/or external to the recipient.

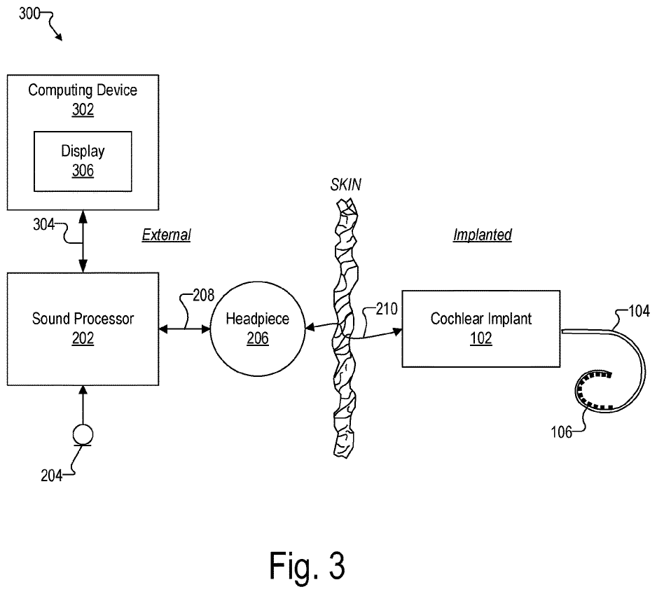

FIG. 3 shows an exemplary configuration 300 of cochlear implant system 100 in which processing unit 108 is implemented by a combination of sound processor 202 and a computing device 302 configured to communicatively couple to sound processor 202 by way of a communication link 304, which may be implemented by any suitable wired or wireless communication link.

Computing device 302 may be implemented by any suitable combination of hardware and software. To illustrate, computing device 302 may be implemented by a mobile device (e.g., a mobile phone, a laptop, a tablet computer, etc.), a desktop computer, and/or any other suitable computing device as may serve a particular implementation. As an example, computing device 302 may be implemented by a mobile device configured to execute an application (e.g., a “mobile app”) that may be used by a user (e.g., the recipient, a clinician, and/or any other user) to control one or more settings of sound processor 202 and/or cochlear implant 102 and/or perform one or more operations (e.g., diagnostic operations) with respect to data generated by sound processor 202 and/or cochlear implant 102.

In some examples, computing device 302 may be configured to control an operation of cochlear implant 102 by transmitting one or more commands to cochlear implant 102 by way of sound processor 202. Likewise, computing device 302 may be configured to receive data generated by cochlear implant 102 by way of sound processor 202. Alternatively, computing device 302 may interface with (e.g., control and/or receive data from) cochlear implant 102 directly by way of a wireless communication link between computing device 302 and cochlear implant 102. In some implementations in which computing device 302 interfaces directly with cochlear implant 102, sound processor 202 may or may not be included in cochlear implant system 100.

Computing device 302 is shown as having an integrated display 306. Display 306 may be implemented by a display screen, for example, and may be configured to display content generated by computing device 302. Additionally or alternatively, computing device 302 may be communicatively coupled to an external display device (not shown) configured to display the content generated by computing device 302.

In some examples, computing device 302 represents a fitting device configured to be selectively used (e.g., by a clinician) to fit sound processor 202 and/or cochlear implant 102 to the recipient. In these examples, computing device 302 may be configured to execute a fitting program configured to set one or more operating parameters of sound processor 202 and/or cochlear implant 102 to values that are optimized for the recipient. As such, in these examples, computing device 302 may not be considered to be part of cochlear implant system 100. Instead, computing device 302 may be considered to be separate from cochlear implant system 100 such that computing device 302 may be selectively coupled to cochlear implant system 100 when it is desired to fit sound processor 202 and/or cochlear implant 102 to the recipient.

Electrodes (e.g., one or more of electrodes 106) electrically coupled to a cochlear implant (e.g., cochlear implant 102) may be used in any suitable manner to measure an evoked response elicited within a recipient of the cochlear implant by stimulation (e.g., electrical stimulation and/or acoustic stimulation). As used herein, an “evoked response” may include any type of cochlear response and/or neural response. Exemplary evoked responses include, but are not limited to, an electrocochleographic (“ECochG”) potential (e.g., a cochlear microphonic potential, a compound action potential such as an auditory nerve response, a summating potential, etc.), a brainstem response (e.g., central auditory potentials of a central auditory pathway of a recipient), a stapedius reflex, and/or any other type of neural or physiological response that may occur within a recipient in response to application of stimulation to the recipient. Evoked responses may originate from neural tissues, hair cell to neural synapses, inner or outer hair cells, and/or other sources.

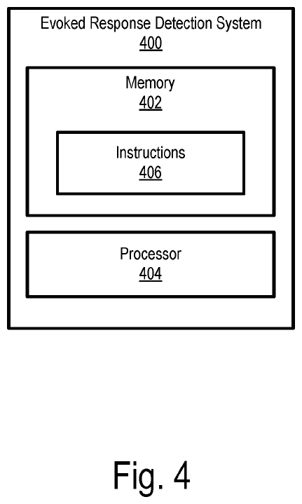

Evoked responses such as those described herein may be used in any suitable manner. For example, evoked responses may be used to fit a hearing device (e.g., a cochlear implant, a hearing aid, etc.) to a recipient, to evaluate the recipient's hearing capability, and/or to monitor auditory brain maturation (e.g., pediatric brain development in relation to the effectiveness of a cochlear implant). To that end, systems and methods such as those described herein are configured to detect an evoked response elicited within a recipient due to stimulation. FIG. 4 illustrates an exemplary evoked response detecting system 400 (“system 400”) that may be implemented according to principles described herein. As shown, system 400 may include, without limitation, a memory 402 and a processor 404 selectively and communicatively coupled to one another. Memory 402 and processor 404 may each include or be implemented by hardware and/or software components (e.g., processors, memories, communication interfaces, instructions stored in memory for execution by the processors, etc.). In some examples, memory 402 and/or processor 404 may be implemented by any suitable computing device such as described herein. In other examples, memory 402 and/or processor 404 may be distributed between multiple devices and/or multiple locations as may serve a particular implementation. Illustrative implementations of system 400 are described herein.

Memory 402 may maintain (e.g., store) executable data used by processor 404 to perform any of the operations described herein. For example, memory 402 may store instructions 406 that may be executed by processor 404 to perform any of the operations described herein. Instructions 406 may be implemented by any suitable application, software, code, and/or other executable data instance.

Memory 402 may also maintain any data received, generated, managed, used, and/or transmitted by processor 404. Memory 402 may store any other suitable data as may serve a particular implementation. For example, memory 402 may store hearing loss profile data, recipient data (e.g., age at implantation, duration of implantation, etc.), evoked response data, electrode position data, dipole data (e.g., dipole location, orientation, polarity, etc.), setting data, stimulation parameter data, graphical user interface content, and/or any other suitable data.

Processor 404 may be configured to perform (e.g., execute instructions 406 stored in memory 402 to perform) various processing operations associated with identifying an evoked response measured through a cochlear implant. For example, processor 404 may perform one or more operations described herein to direct a cochlear implant to continuously record, during an acquisition time window and using the at least one electrode electrically coupled to the cochlear implant, data representative of an evoked response that occurs within a recipient of the cochlear implant in response to stimulation applied to the recipient, and stream, by way of a back telemetry channel, the data to a computing device external to the recipient during the acquisition time window while the cochlear implant is continuously recording the data. These and other operations that may be performed by processor 404 are described herein.

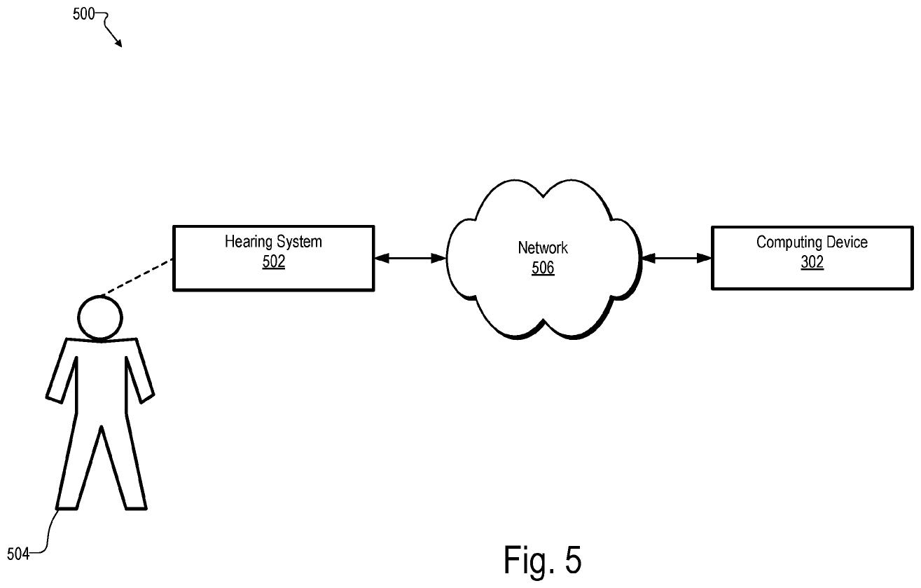

System 400 may be implemented in any suitable manner. For example, FIG. 5 shows an exemplary implementation 500 of system 400 where a hearing system 502 of a recipient 504 is communicatively coupled to computing device 302 by way of a network 506. Hearing system 502 may be implemented by any suitable hearing device or combination of hearing devices as may serve a particular implementation.

As used herein, a “hearing device” may be implemented by any device or combination of devices configured to provide or enhance hearing to a user. For example, a hearing device may be implemented by a cochlear implant system such as described herein. In addition, a hearing device may include hearing aid configured to amplify audio content to a recipient, a sound processor included in a stimulation system configured to apply electrical and/or acoustic stimulation to a recipient, an additional cochlear implant, or any other suitable hearing prosthesis. In some examples, a hearing device may be implemented by a behind-the-ear (“BTE”) housing configured to be worn behind an ear of a user. In some examples, a hearing device may be implemented by an in-the-ear (“ITE”) component configured to at least partially be inserted within an ear canal of a user. In some examples, a hearing device may include a combination of an ITE component, a BTE housing, and/or any other suitable component.

In certain examples, hearing devices such as those described herein may be implemented as part of an electro-acoustic stimulation (“EAS”) system. Such an EAS system combines the functionality of a hearing aid and a cochlear implant system together in the same ear by providing acoustic stimulation representative of low frequency audio content and electrical stimulation representative of high frequency content.

In certain examples, hearing devices such as those described herein may be implemented as part of a binaural hearing system. Such a binaural hearing system may include a first hearing device associated with a first ear of a user and a second hearing device associated with a second ear of a user. In such examples, the hearing devices may each be implemented by any type of hearing device configured to provide or enhance hearing to a user of a binaural hearing system. In some examples, the hearing devices in a binaural system may be of the same type. For example, the hearing devices may each be cochlear implant devices. In certain alternative examples, the hearing devices may be of a different type. For example, a first hearing device may be a hearing aid and a second hearing device may be a sound processor included in a cochlear implant system.

Network 506 may include, but is not limited to, one or more wireless networks (Wi-Fi networks), wireless communication networks, mobile telephone networks (e.g., cellular telephone networks), mobile phone data networks, broadband networks, narrowband networks, the Internet, local area networks, wide area networks, and any other networks capable of carrying data and/or communications signals between hearing system 502 and computing device 302. In certain examples, network 506 may be implemented by a Bluetooth protocol (e.g., Bluetooth Classic, Bluetooth Low Energy (“LE”), etc.) and/or any other suitable communication protocol to facilitate communications between hearing system 502 and computing device 302. In certain examples, network 506 may include a back telemetry channel that communicatively couples a cochlear implant of hearing system 502 to computing device 302 and/or any other suitable device/system (e.g., external components of a cochlear implant system). Communications between hearing system 502, computing device 302, and any other device/system may be transported using any one of the above-listed networks, or any combination or sub-combination of the above-listed networks.

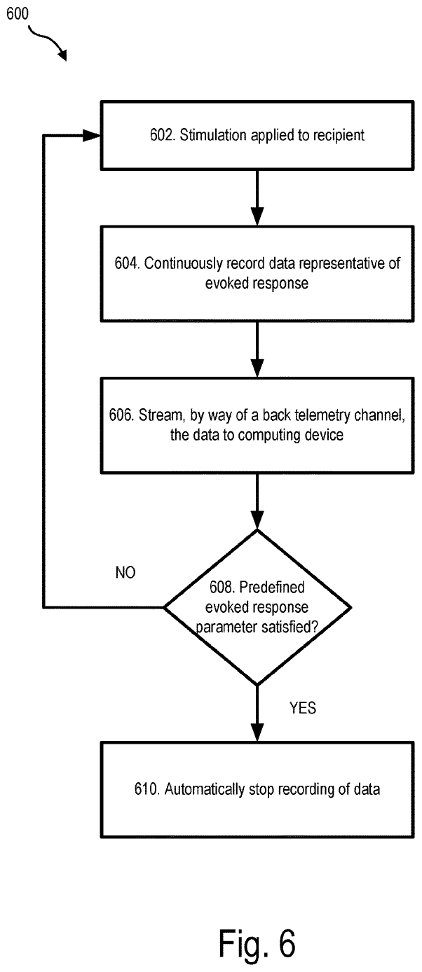

FIG. 6 depicts an exemplary flow diagram 600 that depicts various operations that may be performed by system 400 (e.g., processor 404) to identify an evoked response measured through a cochlear implant. As shown in FIG. 6, at operation 602 stimulation may be applied to a recipient of a cochlear implant to elicit an evoked response. The stimulation may be applied in any suitable manner as may serve a particular implementation. For example, the stimulation may be applied as a pulse train, a modulated pulse train, or any other suitable type of stimulation.

In certain examples, the stimulation may include acoustic stimulation delivered to an ear canal of the recipient. For example, system 400 may direct a receiver of an ITE hearing device of hearing system 502 to provide acoustic stimulation to the recipient. Additionally or alternatively, an acoustic stimulation device that is external to hearing system 502 may be used to provide the acoustic stimulation to the recipient. For example, computing device 302 may include or otherwise be communicatively coupled to a microphone configured to provide acoustic stimulation to the recipient during a fitting session with a clinician.

In certain examples, the stimulation may include electrical stimulation applied to the recipient to elicit an evoked response. Such electrical stimulation may be applied in any suitable manner using any suitable electrode or combination of electrodes. In certain examples, electrical stimulation may be applied by way of one or more electrodes on an electrode lead at least partially inserted within a cochlea of a recipient. The stimulating electrodes may include monopolar electrodes, bipolar electrodes, or any suitable combination of electrodes.

In certain examples, the stimulation may include a combination of electrical stimulation and acoustic stimulation. For example, electrical stimulation may be applied by way of an electrode coupled to a cochlear implant concurrently with acoustic stimulation provided by way of a speaker in a vicinity of an ear of the recipient.

At operation 604, system 400 may continuously record data representative of an evoked response. This may be accomplished in any suitable manner. For example, system 400 may direct a cochlear implant to continuously record, during an acquisition time window and using at least one electrode electrically coupled to the cochlear implant, the data representative of the evoked response. As used herein, “continuously record” may mean that the cochlear implant is in a recording mode during the entire acquisition time window such that the electrodes are used to record evoked responses any time within the acquisition time window. In certain examples, the “continuously recording” may include minimal gaps as long as the gaps do not shadow/void any evoked response characteristics of interest in a meaningful way. The continuously recording of the data may include measuring a signal between the at least one electrode and an additional electrode electrically coupled to the cochlea implant. For example, one or more of electrodes 106 on electrode lead 104 may be used as measuring electrodes and a ring electrode on electrode lead 104 or a ground electrode on cochlear implant 102 may be used as a return electrode. The acquisition time window may have any suitable duration as may serve a particular implementation.

In certain examples, the continuously recording of the data may be initiated prior to the stimulation being applied to the recipient. For example, in implementations where contra lateral recording (e.g., where recording occurs at a first ear of a recipient and stimulation is provided at a second ear) is implemented, system 400 may direct the cochlea implant to start recording before the stimulation is started.

In certain examples, a plurality of electrodes may be used to record the data during the acquisition time window. In such examples, the plurality of electrodes may be used to record the data in any suitable manner. For example, the recording may be done simultaneously with two or more electrodes or sequentially. The recording electrodes may be monopolar electrodes, bipolar electrodes, and/or any suitable combination of electrodes. In certain examples, multichannel recording may be used for artifact cancellation.

System 400 may use any suitable electrode or combination of electrodes to record the data as may serve a particular implementation. For example, system 400 may use a first electrode on an electrode lead of a cochlear implant and a second electrode on a case of the cochlear implant to record the data. In certain examples, the electrodes used to record the data may be on a different side of a recipient than where the stimulation is applied to the recipient. For example, the stimulation may be applied to a first ear of the recipient and the electrode(s) used to record the data may be located at a second ear of the user. In certain alternative implementations, the electrode(s) used to record the data may be located on a same side of the recipient where the stimulation is applied. For example, a first electrode may be located within a first cochlea of a first ear of the recipient, a second electrode may be located within a second cochlea of a second ear of the recipient, and the stimulation (e.g., acoustic stimulation) may be applied to the first ear of the recipient.

At operation 606, system 400 may stream, by way of a back telemetry channel, the data to a computing device that is external to the recipient. For example, system 400 may direct a cochlear implant to stream the data by way of the back telemetry channel to computing device 302 during the acquisition time window. The data may be streamed to the computing device by way of the back telemetry channel in any suitable manner. For example, in certain implementations, the data may be streamed directly from the cochlear implant to the computing device by way of the back telemetry channel. In certain alternative implementations, the data may be streamed to a headpiece of the cochlear implant system and the headpiece may then transmit the data to the computing device in any suitable manner using any suitable network such as described herein.

In certain examples, the cochlear implant may not store the data in a buffer of the cochlear implant prior to streaming the data to the computing device. Rather, the data may be continuously streamed to the computing device by way of the back telemetry channel in real time or near real time as the data is measured by way of the electrodes. By continually recording the data and simultaneously streaming the data by way of the back telemetry channel, it is possible to measure evoked responses for relatively longer durations than conventional methods, which typically require transferring data in small batches due to a relatively small size of a buffer of a cochlear implant.

At operation 608, system 400 may determine whether a predefined evoked response parameter is satisfied. The predefined evoked response parameter may correspond to any suitable parameter that may indicate that the data includes an evoked response. For example, the predefined evoked response parameter may correspond to a peak in the data that has an amplitude at or above a predefined amplitude.

If the answer at operation 608 is “NO,” the flow may return to operation 602 where additional stimulation may be applied to the recipient in any suitable manner such as described herein. System 400 may repeat operations 602-608 any suitable number of times until either a predefined evoked response parameter is satisfied, or the process is stopped in some other manner (e.g., manually by a clinician).

If the answer at operation 608 is “YES,” the flow may proceed to operation 610 where system 400 may automatically stop recording the data. As used herein, the expression “automatically” means that an operation (e.g., a stop recording operation) or series of operations are performed without requiring further input from a user. For example, system 400 may automatically identify and/or label one or more peaks associated with an evoked response without requiring input by a user (e.g., a clinician). In doing so, system 400 may be configured to reduce or eliminate the burden that a clinician may have in determining when to stop measurement of an evoked response and/or in evaluating one or more properties of the evoked response.

In certain examples, system 400 may use additional information to maximize evoked responses measured during an acquisition time window. For example, system 400 may use electrode position information, recipient information (e.g., age at implantation, time since implantation, duration of hearing loss, history of measurements, etc.), stimulation pattern information, measurements indicative of neural survival/health at a stimulation site and/or a site of neural excitation (proximal vs. distal processes of spiral ganglion cells), and/or any other suitable information to maximize evoked responses measured during an acquisition time window. For example, a different stimulation pattern may result in a different evoked response and/or originator. In certain examples, system 400 may select, from a plurality of possible stimulation patterns, a stimulation pattern that generates an optimal evoked response. This may be accomplished in any suitable manner. For example, system 400 may measure responses to different stimulation patterns, determine one or more response characteristics of the measured responses, and select a stimulation pattern to use based on the determined one or more response characteristics. In certain examples, the determining of the one or more response characteristics may include determining neurometric/psychometric functions and/or tuning curves based on stimulation parameters such as the electrode(s) used, pulse rate, pulse width, phase asymmetry (C/A phase durations), amplitude modulation rate/range, pulse width modulation rate/range, stimulation rate modulation rate/range, etc. In certain examples, the selecting of the stimulation pattern may include maximizing/minimizing the characteristic(s) and/or maximizing a similarity of the characteristic(s) to one or more predefined target values. In certain examples, system 400 may alter the stimulation pattern used to avoid adaptation.

Additionally or alternatively, system 400 may implement amplitude modulation, multi-electrode stimulation, and/or any other suitable methodology to optimize evoked responses measured by way of a cochlear implant.

In certain examples, system 400 may be configured to process the data to automatically determine one or more properties of an evoked response. The one or more properties of the evoked response may include any suitable property that may be used to identify and/or evaluate an evoked response. For example, the one or more properties may include one or more peaks, amplitudes of peaks, la and/or any other suitable property.

System 400 may process the data in any suitable manner. For example, system 400 may process the data by comparing the data to additional data representative of a zero-stimulus condition of the recipient. The zero-stimulus condition may capture both biological factors unrelated to the stimulation and biases inherent in the measurement apparatus. System 400 may determine the evoked response of the recipient based on data comparison to a predetermined (default) data (e.g. waveform). In certain examples, the comparing of the data to the additional data may include subtracting portions of the predetermined data from the recorded data to identify the evoked response.

In certain examples, the data may include significant background noise that may need to be removed to adequately identify the evoked response. In such examples, system 400 may determine the background noise in the data and may extract the one or more properties of the evoked response from the background noise. This may be accomplished in any suitable manner. For example, system 400 may use data associated with the zero-stimulus condition to extract the evoked response from the background data. In such examples, system 400 may detect features in data captured during a zero-stimulus condition that statistically differ from the corresponding features during stimulation. For example, the response amplitude at time points after stimulation onset may be compared in real time or during post processing between the stimulation condition and the zero-stimulus condition to identify times where the differences satisfy a predefined condition. In certain examples, additional or alternative features may be used for comparing data captured during stimulation with data captured during the zero-stimulus condition. For example, coefficients of wavelet decomposition and/or inter-trial coherence may be used in certain examples.

In certain examples, system 400 may be configured to automatically extract key elements of the evoked response from the features identified as significantly different between the stimulation condition and the zero-stimulus condition. For example, amplitude and/or latency of a peak may be extracted from time points at which the amplitude of the stimulation condition was significantly different from the zero-stimulus condition.

In certain examples, system 400 may use prior knowledge about population distribution of evoked response features to constrain a detection of an evoked response. For example, the prior knowledge may indicate that the one or more evoked responses are expected to occur within a specific latency range. Accordingly, system 400 may focus on the specific latency range when detecting whether one or more evoked responses are present in the data.

In certain examples, system 400 may select which electrodes to use to record data associated with an evoked response from a plurality of available electrodes. System 400 may select which electrodes to use in any suitable manner. For example, in certain implementations, system 400 may select which electrodes to use based on a position of the electrodes in relation to a dipole orientation of an evoked response originator. In such examples, system 400 may obtain or otherwise use information indicating a physical position of the electrodes to determine which electrodes to use. The physical position of the electrodes may be identified in any suitable manner. For example, a computerized tomography (“CT”) scan may be used in certain examples to determine the positions of electrodes coupled to a cochlear implant. In examples where a case electrode is used, the location of the case electrode may be estimated based on a headpiece location. The orientation of the dipole may be determined in any suitable manner. For example, a scalp electroencephalogram (“EEG”) may be used to identify a dipole size and/or orientation. System 4000 may select the electrodes to be used in the recording of the data by comparing a cochlear implant recorded EEG with a scalp recorded EEG to determine which electrodes provide optimal data (e.g., maximum amplitude, similar morphology, etc.).

In certain examples, system 400 may concurrently use multiple electrodes to record data associated with an evoked response. This may be accomplished in any suitable manner. For example, system 400 may concurrently short two or more electrodes at the same time such that the two or more electrodes functionally operate as a relatively larger electrode contact during recording. In so doing, it may be possible for system 400 to reduce the measurement impedance and have a larger recording radius (which facilitates picking up bigger signals and/or signals that are farther away), thus leading to a relatively larger signal to noise ratio.

In certain examples, system 400 may track originator orientation for rehab purposes.

After processing the data, system 400 may be configured to output the data in any suitable format to be evaluated by a clinician. For example, the data may be output in a chart (e.g., by way of computing device 302) that includes any suitable information associated with the evoked response(s). In certain examples, the chart may include a central auditory potential waveform with one or more automatically identified peaks representing central auditory potentials of a central auditory pathway of the recipient.

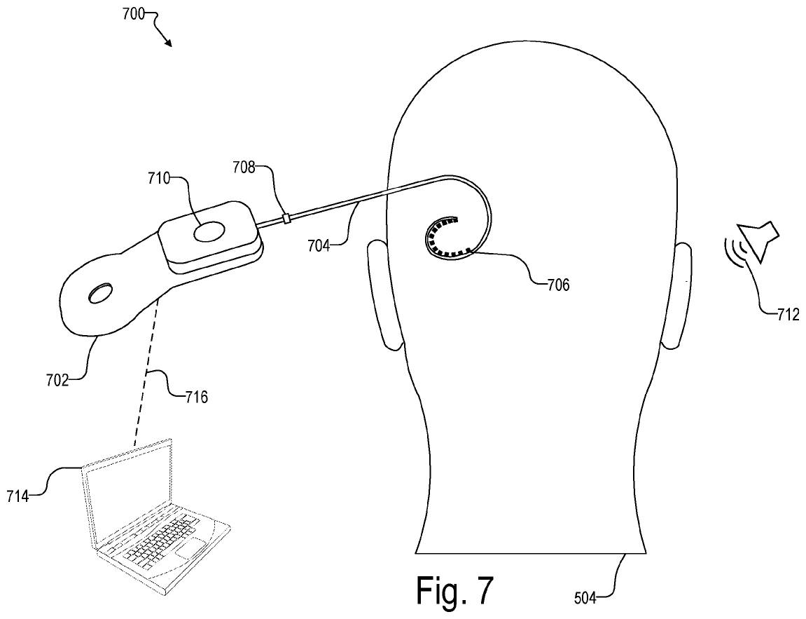

FIGS. 7-10 illustrate exemplary configurations 700-1000 in which system 400 may be implemented in certain examples. In configuration 700 shown in FIG. 7, a cochlear implant 702 is depicted in relation to a head of recipient 504. FIG. 7 shows cochlear implant 702 as being partially apart from the head of recipient 504 for illustrative purposes. It is understood that cochlear implant 702 is implanted within recipient 504. As shown in FIG. 7, cochlear implant 702 is electrically coupled to an electrode lead 704 that includes electrodes 706 on a distal end and a ring electrode 708 on a proximal end towards cochlear implant 702. Cochlear implant 702 further includes a case electrode 710. As shown in FIG. 7, stimulation 712 (e.g., acoustic stimulation) may be provided to the ear on the right side of the head. Any suitable combination of electrodes 706, ring electrode 708, and/or case electrode 710 may be used by system 400 to continuously record data during a data acquisition window. Cochlear implant 702 may stream the data to a computing device 714 during the data acquisition window by way of a back telemetry channel 716. Computing device 714 is shown as a laptop computer in FIG. 7 but could be implemented by any other suitable computing device such as described herein.

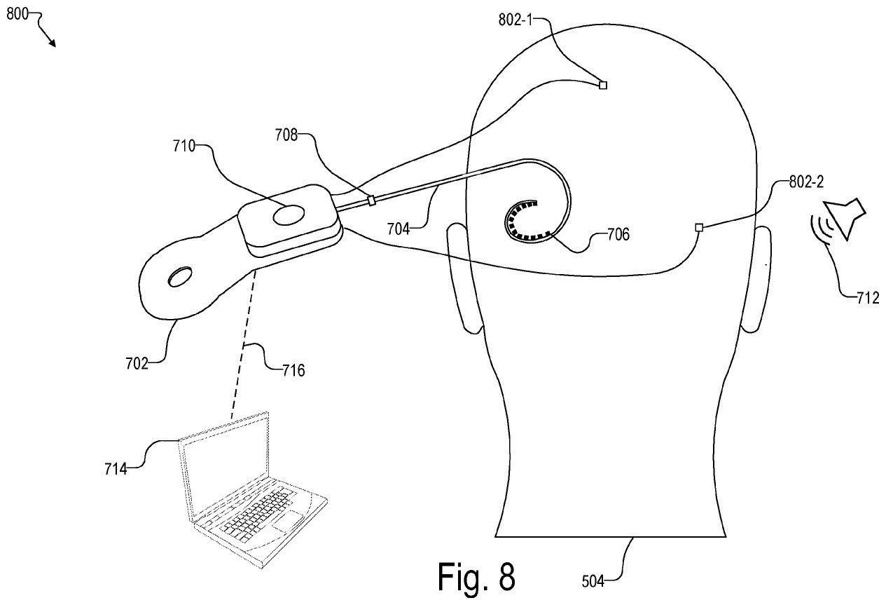

In certain examples, a cochlear implant may include one or more additional electrodes specifically configured to be used to record data associated with an evoked response. To illustrate an example, configuration 800 shown in FIG. 8 is similar to configuration 700 shown in FIG. 7 except that additional electrodes 802 (e.g., electrodes 802-1 and 802-2) are electrically coupled to cochlear implant 702. In the example shown in FIG. 8 any suitable combination of electrodes 802, electrodes 706, ring electrode 708, and/or case electrode 710 may be used by system 400 to continuously record data during a data acquisition window.

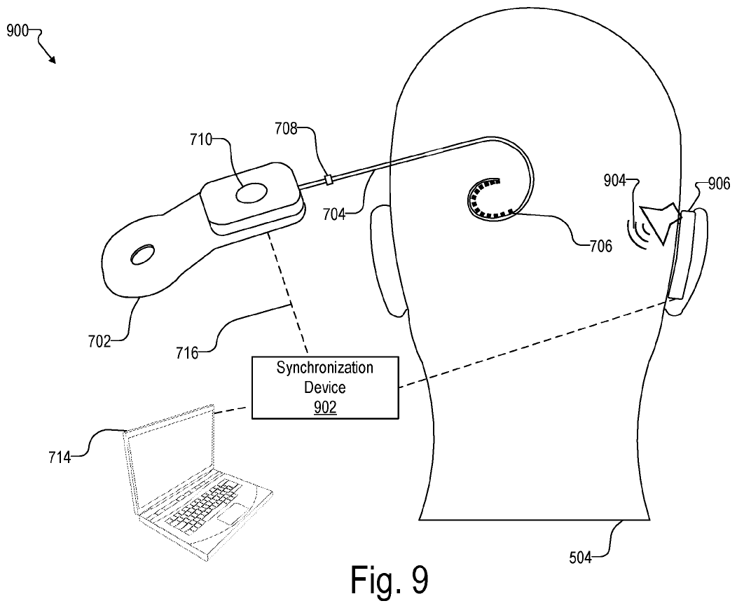

In certain examples, system 400 may use a synchronization device to synchronize the stimulation provided to elicit an evoked response and the recording of the data. The synchronization device may be used in any suitable manner to synchronize the stimulation. For example, the synchronization device may be used to synchronize the stimulation and recording; to synchronize stimulation/recording from a device in one ear to the stimulation/recording on device in an opposite ear; and/or to synchronize stimulation/recording in one device to stimulation/recording on an external device/system (e.g., speakers/Bluetooth audio, and/or an external EEG system). FIG. 9 shows an exemplary configuration 900 where a synchronization device 902 is provided to synchronize stimulation 904 provided by way of a BTE hearing aid 906 with the recording of the data by cochlear implant 702. Synchronization device 902 may be implemented in any suitable manner. For example, synchronization device 902 may be implemented by a separate physical device communicatively coupled with BTE hearing aid 906 and cochlear implant 702. In certain alternative implementations, synchronization device 902 may be implemented by software executed by computing device 714.

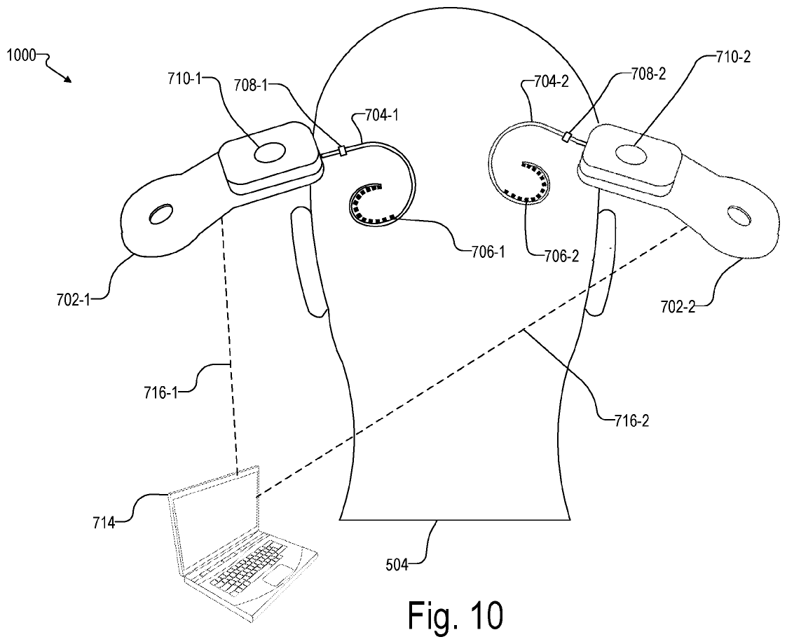

FIG. 10 illustrates an exemplary configuration 1000 in which cochlear implants 702 (e.g., cochlear implant 702-1 and cochlear implant 702-2) are provided at each ear of recipient 504. In the example shown in FIG. 10, electrical stimulation may be provided, for example, by way of one or more of electrodes 706-2. Any suitable combination of the electrodes coupled to cochlear implant 702-1 or electrodes coupled to cochlear implant 702-2 may be used to record data representative of an evoked response that occurs in response to the electrical stimulation. Data representative of the evoked response elicited by the stimulation may be streamed to computing device 714 by way of back telemetry channel 716-1 concurrently with the stimulation.

Although the preceding description is described in the context of a cochlear implant system, it is understood that concepts such as those described herein may be applied in other contexts with other types of implantable electrode leads.

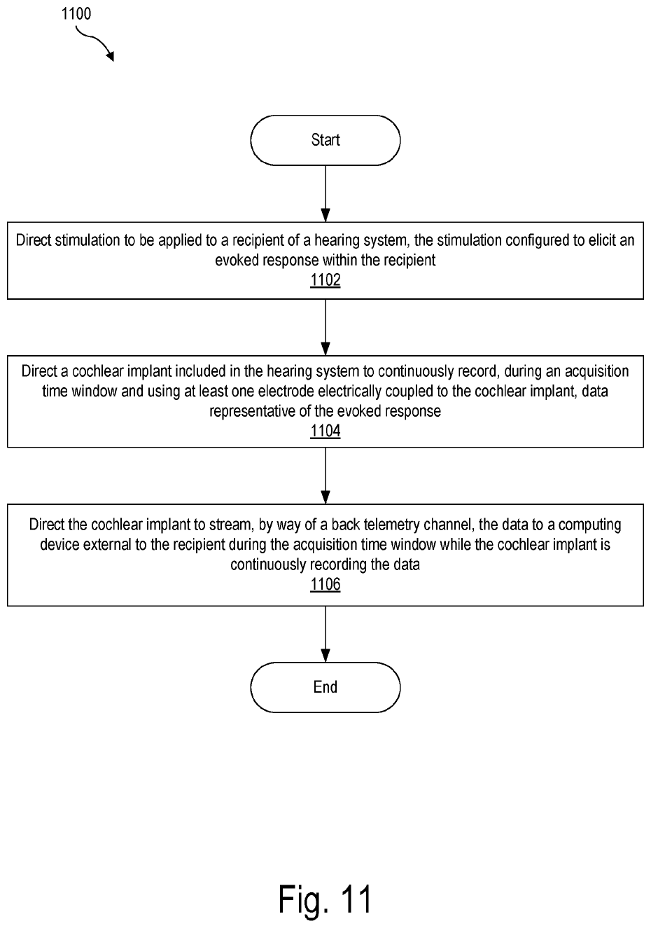

FIG. 11 illustrates an exemplary method 1100 for identifying an evoked response measured through a cochlear implant. While FIG. 11 illustrates exemplary operations according to one embodiment, other embodiments may omit, add to, reorder, and/or modify any of the operations shown in FIG. 11.

At operation 1102, an evoked response detection system (e.g., evoked response detection system 400) may direct stimulation to be applied to a recipient of a hearing system. The stimulation may be configured to elicit an evoked response within the recipient. Operation 1102 may be performed in any of the ways described herein.

At operation 1104, the system may direct a cochlear implant included in the hearing system to continuously record, during an acquisition time window and using at least one electrode electrically coupled to the cochlear implant, data representative of the evoked response. Operation 1104 may be performed in any of the ways described herein.

At operation 1106, the system may direct the cochlear implant to stream, by way of a back telemetry channel, the data to a computing device that is external to the recipient during the acquisition time window while the cochlear implant is continuously recording the data. Operation 1106 may be performed in any of the ways described herein.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g., a hard disk, a floppy disk, magnetic tape, etc.), ferroelectric random-access memory (RAM), and an optical disc (e.g., a compact disc, a digital video disc, a Blu-ray disc, etc.). Exemplary volatile storage media include, but are not limited to, RAM (e.g., dynamic RAM).

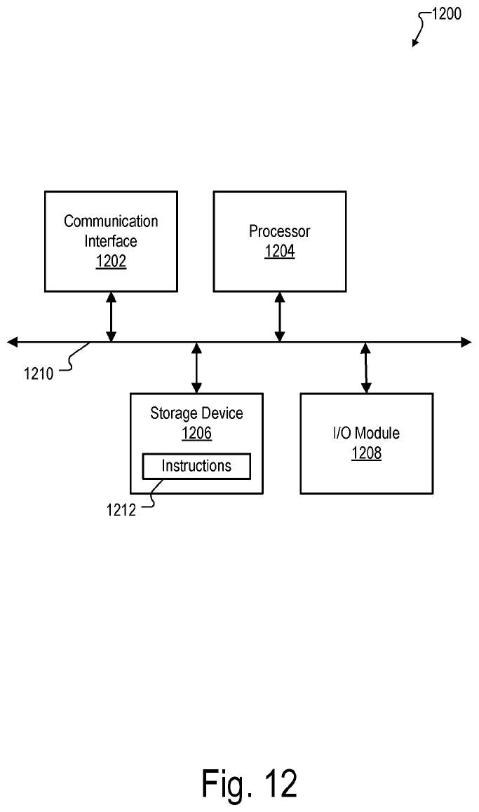

FIG. 12 illustrates an exemplary computing device 1200 that may be specifically configured to perform one or more of the processes described herein. As shown in FIG. 12, computing device 1200 may include a communication interface 1202, a processor 1204, a storage device 1206, and an input/output (I/O) module 1208 communicatively connected one to another via a communication infrastructure 1210. While an exemplary computing device 1200 is shown in FIG. 12, the components illustrated in FIG. 12 are not intended to be limiting. Additional or alternative components may be used in other embodiments. Components of computing device 1200 shown in FIG. 12 will now be described in additional detail.

Communication interface 1202 may be configured to communicate with one or more computing devices. Examples of communication interface 1202 include, without limitation, a wired network interface (such as a network interface card), a wireless network interface (such as a wireless network interface card), a modem, an audio/video connection, and any other suitable interface.

Processor 1204 generally represents any type or form of processing unit capable of processing data and/or interpreting, executing, and/or directing execution of one or more of the instructions, processes, and/or operations described herein. Processor 1204 may perform operations by executing computer-executable instructions 1212 (e.g., an application, software, code, and/or other executable data instance) stored in storage device 1206.

Storage device 1206 may include one or more data storage media, devices, or configurations and may employ any type, form, and combination of data storage media and/or device. For example, storage device 1206 may include, but is not limited to, any combination of the non-volatile media and/or volatile media described herein. Electronic data, including data described herein, may be temporarily and/or permanently stored in storage device 1206. For example, data representative of computer-executable instructions 1212 configured to direct processor 1204 to perform any of the operations described herein may be stored within storage device 1206. In some examples, data may be arranged in one or more databases residing within storage device 1206.

I/O module 1208 may include one or more I/O modules configured to receive user input and provide user output. One or more I/O modules may be used to receive input for a virtual experience. I/O module 1208 may include any hardware, firmware, software, or combination thereof supportive of input and output capabilities. For example, I/O module 1208 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1208 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1208 is configured to provide graphical data to a display for presentation to a user. The graphical data may be representative of one or more graphical user interfaces and/or any other graphical content as may serve a particular implementation. In certain embodiments, I/O module 1208 may be configured to provide a trigger signal associated with any of the operations described herein. For example, the trigger signal provided by I/O module 1208 may be used to facilitate detecting evoked responses during bilateral stimulation.

In some examples, any of the systems, computing devices, and/or other components described herein may be implemented by computing device 1200. For example, memory 112 or memory 402 may be implemented by storage device 1206, and processor 114 or processor 404 may be implemented by processor 1204.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A system comprising:
a memory storing instructions; and
a processor configured to execute the instructions to perform a process comprising:
directing stimulation to be applied to a recipient of a hearing system, the stimulation configured to elicit an evoked response within the recipient;
directing a cochlear implant included in the hearing system to continuously record, during an acquisition time window and using at least one electrode electrically coupled to the cochlear implant, data representative of the evoked response; and
directing the cochlear implant to stream, by way of a back telemetry channel, the data to a computing device external to the recipient during the acquisition time window while the cochlear implant is continuously recording the data.

2. The system of claim 1, wherein the stimulation is at least one of acoustic stimulation or electrical stimulation.

3. The system of claim 1, wherein the stimulation is provided at a first ear of the recipient and the at least one electrode is positioned at a second ear of the recipient.

4. The system of claim 1, wherein the at least one electrode is located on an electrode lead connected to the cochlear implant and at least partially inserted within a cochlea of the recipient.

5. The system of claim 1, wherein the continuously recording of the data includes measuring a signal between the at least one electrode and an additional electrode electrically coupled to the cochlear implant.

6. The system of claim 1, wherein:
the at least one electrode is located at a first ear of the recipient; and
the continuously recording of the data includes measuring a signal between the at least one electrode and at least one additional electrode electrically coupled to an additional cochlear implant located at a second ear of the recipient.

7. The system of claim 1, wherein the continuously recording of the data is initiated prior to the stimulation being applied to the recipient.

8. The system of claim 1, wherein the evoked response includes central auditory potentials of a central auditory pathway of the recipient.

9. The system of claim 1, wherein the process further comprises processing the data to determine one or more properties of the evoked response.

10. The system of claim 9, wherein the processing of the data comprises:
determining background noise in the data; and
extracting the one or more properties of the evoked response from the background noise.

11. The system of claim 9, wherein the processing of the data comprises:
comparing the data to additional data representative of a zero-stimulus condition of the recipient; and
determining the evoked response of the recipient based on the comparing of the data to the additional data.

12. The system of claim 9, wherein the process further comprises:
determining that the evoked response satisfies a predefined evoked response parameter; and
automatically stopping, based on the determining that the evoked response satisfies the predefined evoked response parameter, the continuously recording of the data.

13. The system of claim 9, wherein the process further comprises:
determining that the evoked response does not satisfy a predefined evoked response parameter;
directing, based on the determining that the evoked response does not satisfy the predefined evoked response parameter, additional stimulation to be applied to the recipient, the additional stimulation configured to elicit an additional evoked response within the recipient;
directing the cochlear implant to continuously record, during an additional acquisition time window and using the at least one electrode, additional data representative of the additional evoked response; and
directing the cochlear implant to stream, by way of the back telemetry channel, the additional data to the computing device external to the recipient during the additional acquisition time window while the cochlear implant is continuously recording the additional data.

14. The system of claim 13, wherein the process further comprises processing the additional data to determine one or more properties of the additional evoked response.

15. A system comprising:
a cochlear implant; and
at least one electrode electrically coupled to the cochlear implant,
wherein the cochlear implant is configured to:
continuously record, during an acquisition time window and using the at least one electrode electrically coupled to the cochlear implant, data representative of an evoked response that occurs within a recipient of the cochlear implant in response to stimulation applied to the recipient; and
stream, by way of a back telemetry channel, the data to a computing device external to the recipient during the acquisition time window while the cochlear implant is continuously recording the data.

16. The system of claim 15, wherein the at least one electrode is located on an electrode lead connected to the cochlear implant and at least partially inserted within a cochlea of the recipient.

17. The system of claim 15, wherein:
the at least one electrode is located at a first ear of the recipient;
the cochlear implant is communicatively coupled to a hearing device located at a second ear of the recipient; and
the stimulation is provided to the recipient by way of the hearing device.

18. The system of claim 17, wherein the system further comprises a synchronization device that is configured to synchronize the stimulation with the continuously recording of the data.

19. A method comprising:
directing, by an evoked response detection system, stimulation to be applied to a recipient of a hearing system, the stimulation configured to elicit an evoked response within the recipient;
directing, by the evoked response detection system, a cochlear implant included in the hearing system to continuously record, during an acquisition time window and using at least one electrode electrically coupled to the cochlear implant, data representative of the evoked response; and
directing, by the evoked response detection system, the cochlear implant to stream, by way of a back telemetry channel, the data to a computing device external to the recipient during the acquisition time window while the cochlear implant is continuously recording the data.

20. The method of claim 19, further comprising processing, by the evoked response detection system, the data to determine one or more properties of the evoked response.
